(19) United States
(12) Patent Application Publication
(10) Pub. No.: US 2019/0282725 A1
(43) Pub. Date: Sep. 19, 2019

(54) SANITARY AID WITH ANTIBACTERIAL EFFECT FOR STABILIZATION OF PHYSIOLOGICAL BALANCE IN VAGINAL ENVIRONMENT AND/OR EXTERNAL GENITALIA

(71) Applicant: FIDE s.r.o., Brtnice (CZ)

(72) Inventors: Dominika MOCUBOVA, Pribyslav (CZ); Libor MADERA, Batelov (CZ)

(21) Appl. No.: 15/924,355

(22) Filed: Mar. 19, 2018

(51) Int. Cl. A61L 15/44; A61L 15/60; A61L 15/28; A61F 13/84; A61F 13/53; A61F 13/20; B32B 5/02; B32B 29/00; D21H 19/20; D21H 27/00

(52) U.S. Cl. CPC A61L 15/44; B32B 2555/00; A61L 15/28; A61F 13/8405; A61F 13/53; A61F 13/2074; B32B 5/022; B32B 29/005; D21H 19/20; D21H 27/002; A61L 2300/404; A61F 2013/530481; B32B 2255/02; B32B 2255/12; B32B 2307/726; B32B 2307/7145; B32B 2555/02; A61L 15/60

(57) ABSTRACT

A sanitary aid with antibacterial effect for stabilization of physiological balance in vaginal environment and/or external genitalia is made by a backsheet impermeable layer, absorbent core formed by a mixture of absorbent and/or superabsorbent and a topsheet layer from woven or non-woven textile or from cellulose. The sanitary aid contains an effective component on the basis of at least one reactive form of oxygen, which is encapsulated by an encapsulation agent, where the effective substance is arranged on the topsheet layer of the sanitary aid designated for the contact with body and/or in the absorbent core. The sanitary aid is adapted for gradual release of reactive forms of oxygen, particularly hydrogen peroxide after contact with liquid from the sanitary aid, which causes the prolongation of the effect of reactive forms of oxygen with substantial disinfection ability.

### FIELD OF THE INVENTION

Technical solution is related to the area of intimacy hygiene, specifically a sanitary aid with antibacterial effect for stabilization of physiological balance in vaginal environment and/or external genitalia.

### BACKGROUND OF THE INVENTION

A healthy female urogenital tract is colonized by complex and specific microbial ecosystem with more than 50 various bacterial species. Physiologically this area is typical for lower pH and production of low amount of hydrogen peroxide, which protects against infection and multiplication of pathogenic microorganisms. The composition of micro flora changes during the life of a female. For example, prevailing microorganisms for women in fertile period are bacteria producing lactic acid, which belong in Lactobacillus genus. However, microbial balance may be disturbed by hormonal changes during the cycle or in menopause.

The production of hydrogen peroxide is a significant protective mechanism in the fight against pathogenic microorganisms. In higher concentrations it is toxic for some bacteria and stops their growth. In the physiological vaginal environment of healthy women Lactobacilli producing hydrogen peroxide were isolated, whereas in women suffering from vaginal infections they were usually missing and the Lactobacilli without the ability to produce hydrogen peroxide were prevailing. Low pH is also very important for maintaining healthy micro flora in the entire vaginal area because it has a natural inhibition effect on pathogenic microorganism such as, for instance, yeast of Candida genus, and other unfavorable bacterial families. Thus the common soap and other alkaline products, which are completely unfavorable for external genitalia, should never be used for intimacy feminine hygiene, as they may disturb the natural acid environment of vagina and thus liquidate this significant protection against pathogens. Perfumed soaps, sanitary pads and other products of intimacy hygiene may further irritate sensitive skin and mucous membranes. What is favorable, on the other hand, are the intimacy hygiene products with pH 5.5, which help stabilize pH in the area of external genitalia and thus help prevent the unpleasant infections.

The composition of vaginal micro biome and physiological pH of intimacy parts of a healthy woman may be disturbed by using antibiotics, chemotherapeutics, antimycotics, during hormonal changes in the organism such as pregnancy, menopause, menstruation period, or by using contraceptives, as well as by wearing unsuitable underwear or due to metabolic changes for women suffering from diabetes, etc. The basic anatomy also implies the risk of spreading undesirable microorganism from the rectum area into the urogenital area. The source of potential infection may be also the use of menstrual pads, towels, napkins, or incontinency aids, in which pathogenic microorganisms may be multiplied at the time of use, and then subsequently transferred to urogenital area, thus causing infection. All the mentioned factors may result in disturbance of normal microbial flora, change in physiological pH of vaginal mucous and the external genitalia, subsequently causing microbial infections, which cause inflammation of vagina, urinary tract, and common skin inflammation, particularly intertrigo. The originators of these infections are particularly the microorganism genera Escherichia, Enterococcus, Psedomonas, Proteus, Klebsiella, Streptococcus, Staphylococcus, Gardnerella, Ureplasma, Bacteriodes, Peptococcus, Neisseria, Serratia, Corynebacterium, Clostridium and Candida. The list of represented microorganism genera is significantly influenced by the fact that for women, due to the shorter distance between rectum and urogenital tract, there is an increased risk of migration of these microorganisms from rectum to vagina and external genitalia. The risky group, in terms of threat of infections, is young women and girls who do not have the vaginal micro flora yet completely developed and stabilized, and on the other hand, older women, whose natural micro flora is changed due to hormonal changes. The risk is also increased at any time during the entire life due to temporary hormonal changes and instabilities, which the organism must cope with, and also due to stress situations and as a result of some diseases or medication.

In the fight against unfriendly microorganisms the probiotic cultures themselves produce a significant effective substance—hydrogen peroxide. This substance, belonging among reactive forms of oxygen, is well-known and commonly used for its disinfection effects and in the discussed system it is one of the key contributors for maintaining microbial balance in vaginal environment. It was published, for instance, that the production of hydrogen peroxide by symbiotic micro flora plays an important role in inhibiting the multiplication of yeast at vaginal mucosa. Its natural production is constant and it involves a substantially small amount, which is, however, for healthy micro flora sufficient to inhibit adverse microorganisms. Its safety in low concentrations is proven also by its natural production in urogenital tract as well as by the fact that it is quickly broken down to molecular oxygen and water.

Hydrogen peroxide is effective against a wide spectrum of microorganism—bacteria, yeast, fungi, viruses, and spores. Furthermore, anaerobe microorganisms are significantly more sensitive to effects of hydrogen peroxide due to the fact that they do not produce catalysis, which breaks it down and thus they are not able to impede its effects. The water solution of hydrogen peroxide with the concentration of 3% shows high efficiency against vegetative forms of bacteria. It was found that hydrogen peroxide shows higher efficiency against gram-negative bacteria than against gram-positive bacteria. Its activity is, compared to other disinfection means such as phenols and organic acids, less influenced by pH, which is very important in vaginal environment. The mechanism of effect of hydrogen peroxide lies in the oxidation of lipids in cell membranes, proteins and DNA by highly reactive hydroxyl radical, which is created during the break-down of hydrogen peroxide to oxygen and water.

What is very significant is also its safety as well as the safety of products of its break-down. It does not have carcinogenic and mutagenic effects, does not harm environment and its residues are easily degradable. The diploma thesis of Michaela Kosova named “Antistaphylococ Activity of Lactobacilli” pays closer attention to the abilities of Lactobacillus generus to produce hydrogen peroxide. The data published in this thesis show that all tested strains of Lactobacillus generus formed hydrogen peroxide in the range 0.04-11.37 mg.l⁻¹ of phosphate buffered saline (PBS) and that the most hydrogen peroxide (11.37 mg.l⁻¹ PBS) was produced by the isolate of Lbc. crispatus L2. All tested strains of Lactobacillus generus, particularly the strains isolated from the vaginal tract of women, could be recommended for practical application to vaginal preparations based on the mixed bacterial consortia with proven antistaphylococ effects. This thesis confirms the significance of the hydrogen peroxide for the maintenance of health of vaginal mucosa.

The current market offers a variety of products designated for intimacy care for external genitalia and vagina environment. Their expected positive effect may be based on several principles. Many products target at optimal adjustment of pH of female intimacy parts. These are usually based on a simple application of creams, gels, intimacy soaps containing lactic acid, which is one of the most important natural factors ensuring low pH of vaginal mucosa and external genitalia and thus also the optimum conditions for friendly microorganisms. Another category is formed by various probiotic creams, suppositories, tampons, pads or globules containing probiotic cultures with the objective to compete with the unfriendly micro flora.

As mentioned earlier, under common circumstances hydrogen peroxide in healthy women is produced mainly by bacteria of Lactobacillus genus. Nevertheless, if stability of vaginal microbial flora is disturbed, for instance by an inflammation, there is a reduced production of hydrogen peroxide and lactic acid due to a decline in Lactobacilli, which usually leads to disturbance of optimum level of pH and reduced ability of the environment to inhibit some pathogenic microorganisms. In this situation it would be suitable, next to the mentioned adjustment of pH and addition of friendly micro flora, to locally adjust the production of hydrogen peroxide by its gentle external administration, whether by application in the form of intimacy gel, soap, pads, tampon or other products containing hydrogen peroxide. The problem and the cause why the market has not yet offered this possibility, is the instability of the applied hydrogen peroxide and its premature break-down, often even before the actual use of the product.

The known solution are feminine intimacy hygiene products based on the file CN101328645 (A), which describes a sanitary product containing a ceramic powder, which in combination with other elements generates infrared radiation that has, according to the standpoint of authors, a positive impact on the reduction of inflammations in intimacy parts, based on the principle of inhibition of microbes in urogenital area. Documents KR20110034190 and KR20090119943 deal with the issue of infections of urogenital tract due to the use of menstrual pads releasing oxygen. In this design the sanitary product is infused with a solid form of acid and oxygenerator, which is activated by the acid. The disadvantage of this solution is the necessary presence of the acid. The oxygenator may be for instance magnesium peroxide or calcium peroxide.

U.S. Pat. No. 7,235,263 B2 solves the use of peroxide bound to sugar alcohols, particularly mannitol or sorbitol. This mixture prepared by mixing hydrogen peroxide with sugar alcohols after a long-term boiling at the temperature of 97° C. begins to crystallize and it may be added in absorbents of sanitary aids. The disadvantage is their quick dissolution and breakdown and subsequent quick loss of disinfection effect during the contact with water solutions. The application for invention US 2017/0001864 A1 offers the use of a bound peroxide, which is released as a result of humidity (e.g. in air) for antimicrobial effect. However, it does not deal with the extension or postponement of its release.

The problem of instability of hydrogen peroxide in the intimacy hygiene products and seeking for the solution in the past was subject to the research of the utility model CZ 28161, the basis of which is the sanitary, hygienic, or medical aid with antibacterial effect containing at least one effective substance producing hydrogen peroxide, preferably polyvinylpyrrolidone-hydrogen peroxide. The release of peroxide takes place without regulation. Although this ensures the release of effective substance only after humidification, that is after the use of the hygienic aid, which is a big progress in the field of disinfection of hygienic aids at the time of use, the release of hydrogen peroxide takes place immediately, without regulation, and thus the period of release of the effective substance is very short and does not reach the full time of common use of hygienic aids.

None of the above-mentioned solutions offers extension of the effect of the released peroxide, active oxygen, etc. The objective of the technical solution is thus to create a sanitary aid with antibacterial effect to stabilize physiological balance of vaginal environment and/or external genitalia, which would provide the extension of antimicrobial effect by gradually releasing reactive molecules of oxygen, respectively hydrogen peroxide, which would be effectively stabilized in the sanitary aid, thus increasing their treatment and preventive effects, and as a result extending the antimicrobial effect.

### SUMMARY OF THE INVENTION

The above-mentioned drawbacks are eliminated by a sanitary aid with antibacterial effect for stabilization of physiological balance in vaginal environment and/or external genitalia based on this invention. The sanitary aid is formed by a backsheet impermeable layer, absorbent core formed by a mixture of absorbent and/or superabsorbent and a topsheet layer from woven or non-woven textile or from cellulose, and contains an effective component on the basis of at least one reactive form of oxygen, which is arranged on the topsheet of the sanitary aid designated for the contact with body and/or in the absorbent core. The basis of the invention lies in the fact that the reactive forms of oxygen are contained in the effective substance in the form encapsulated by an encapsulation agent, which allows gradual release of reactive forms of oxygen after the contact with liquid from the sanitary aid, causing the prolongation of the effect of the reactive forms of oxygen with substantial disinfection ability. Reactive forms of oxygen producing particularly hydrogen peroxide are contained in the sanitary aid or medical aid in the form of an encapsulated complex. In addition, the reactive form of oxygen encapsulated in the sanitary aid is stable and the production of hydrogen peroxide and/or active oxygen and/or hydroxyl radicals takes place only after the contact with the liquid, particularly with water, blood, urine, sweat, or discharge, that is only after the beginning of use. Furthermore, due to the treatment by encapsulation by other encapsulation agents, the humidification takes place gradually and as a result the activation of reactive forms of oxygen takes place gradually, and thus the active effect of the disinfection additive is extended, providing longer protection against adverse impacts.

In a preferred embodiment the reactive form of oxygen in the form of organic peroxide complex, particularly polyvinylpyrrolidone-hydrogen peroxide i.e. 2-Pyrrolidinone,1-Ethenyl, homopolymer, Hydrogen Peroxide, or in the form of inorganic hydrogen peroxide compounds, particularly in the form of magnesium peroxide, calcium peroxide, sodium percarbonate and sodium perborate, or their significant combinations.

In addition, the reactive form of oxygen is encapsulated by an encapsulation agent from the following groups: monolaurin, monoglycerides, stearanes, glyceryles, stearates, stearoyls, lauroyls, laurates and other derivatives of fatty acids, microcrystalline cellulose, hydroxypropylmethyl cellulose, omega-3 fatty acids, mannitol, sorbitol, xylitol, isomalt, humin compounds, sunflower oil, rapeseed oil, olive oil, carnauba wax, bee wax, phospholipids, lactic and whey proteins, casein, gelatin, gluten, paraffin, shellac or their mutual combinations.

In a preferred embodiment, reactive form of oxygen is encapsulated in the ratio 0.01 to 99 parts by weight of the reactive form of oxygen and 0.1 to 99.9 parts by weight of the encapsulation agent, preferably 1 to 5 parts by weight of the reactive form of oxygen and 2 to 20 parts by weight of the encapsulation agent.

In a preferred embodiment, effective substance is in the form of micronized particles with the size of 1 to 999 μm, preferably 1 to 500 μm, more preferably 1 to 100 μm.

In a preferred embodiment, the topsheet layer of the sanitary aid contains a mixture formed by 0.01 to 99.99 parts by weight, with an advantage 50 to 90 parts by weight of the absorbent or superabsorbent and 0.01 to 50 parts by weight, with an advantage 5 to 20 parts by weight of the effective substance, that is reactive forms of oxygen with an encapsulation agent gradually releasing hydrogen peroxide.

Effective substance in the absorbent core is preferably formed by a mixture, which contains 1 to 99.999 parts by weight, with an advantage 50 to 99.9 parts by weight of absorbent or superabsorbent and 0.001 to 50 parts by weight, with an advantage 0.1 to 3 parts by weight of the effective substance, that is reactive forms of oxygen with an encapsulation agent gradually releasing hydrogen peroxide. The absorbent core may contain cellulose, carbosymethylcellulose, its derivatives, and/or hydroxymethylcelullose and/or its derivatives, and/or hydroxymethylpropylcelullose and/or its derivatives, and/or other organic absorbent polymers.

The sanitary aid also contains preferably freely arranged reactive forms of oxygen at the topsheet layer of the sanitary aid designated for contact with the body and/or in the internal structure of the sanitary aid.

In a preferred embodiment, the sanitary aid also contains an auxiliary substance in the topsheet layer of the sanitary aid designated for contact with the body and/or in the internal structure of the sanitary aid selected from the group: lactic acid, its pharmaceutically acceptable salt, lyophilized probiotic culture, prebiotic element, plant extracts, preferably herb extracts, titanium dioxide, antimicrobial metals, preferably silver, or their mutual mixtures.

In a preferred embodiment, the sanitary aid is made in the form of menstrual pads, towels or napkins or daytime or overnight pads or panty liners, or baby diapers, or incontinence pads or napkins, or tampons, or toilet paper, or paper tissue.

The sanitary aid with antibacterial effect according to this invention shows treatment, disinfection, antimicrobial and preventive effects and by its composition naturally contributes to stabilization of physiological balance in vaginal mucosa and external genitalia, rectum and skin in intimacy parts, and particularly inhibits the multiplication of adverse micro flora at the sanitary aid itself. The mentioned sanitary aid and the method of gradual release of reactive forms of oxygen from the sanitary aid is sensitive to the body and skin of women due to the gradual and gentle release of active substances and the inhibition of pathogenic micro flora, so it allows natural and spontaneous restoration of optimum healthy micro flora, which may naturally vary for different women. This is all allowed in the extended interval of time, which thus offers the possibility of more effective prevention and higher efficiency in the process of stabilization of microenvironment of the entire intimacy area, and thus also a lower risk or subsequent re-occurrence of infections.

### DETAILED DESCRIPTION OF THE PREFERRED EMBODIMENTS OF THE INVENTION

### Example 1

The effective substance contains a reactive form of oxygen on the basis of 2-Pyrrolidinone,1-Ethenyl-, homopolymer, Hydrogen Peroxide or polyvinylpyrrolidone-hydrogen peroxide with the ability to release hydrogen peroxide, which is encapsulated by an encapsulation agent glyceryl monostrarate by a process of gentle grinding of a stirred mixture in a fragmentation grinder at the laboratory temperature without heating, in weight ratios (1:2, 1:1, 2:1). The encapsulate or the effective substance is homogenized with superabsorbent in the ratio of 1 part of effective substance and 99 parts of superabsorbent. Such prepared mixture is used for the production of an absorbent core of a menstrual pad. The mixture is applied in the absorbent core of a menstrual pad in the amount of 1.5 g.

### Example 2

The effective substance contains a reactive form of oxygen on the basis of 2-Pyrrolidinone,1-Ethenyl-, homopolymer, Hydrogen Peroxide or polyvinylpyrrolidone-hydrogen peroxide with the ability to release hydrogen peroxide, which is encapsulated by an encapsulation agent glyceryl monostearate by a process of homogenization by grinding the mixture in a fragmentation grinder and the subsequent encapsulation or encapsulation in the presence of an organic solvent hexane. After encapsulation or encapsulation the distillation of the mixture was made until dry at the water bath temperature of 25° C. and the size of achieved parts is adjusted by sieving through a sieve with the size of 0.5 mm. The encapsulate or effective element is homogenized with superabsorbent in the ratio of 2 parts by weight of effective substance and 98 parts by weight of superabsorbent. Such prepared mixture is used for the production of an absorbent core of a menstrual pad. The mixture is applied in the absorbent core of a menstrual pad in the amount of 1.3 g.

### Example 3

The effective substance contains a reactive form of oxygen on the basis of sodium perborate with the ability to release hydrogen peroxide, and is encapsulated by an encapsulation agent glyceryl monolaurate in the ratio of 1:3 in an industrial extruder with the regulation of temperature by a process of active cooling for the purpose of protection of reactive form of oxygen against breakdown by heat. Such prepared mixture is applied by a roller on the internal side of the topsheet from non-woven textiles in the amount of 1 g per 1 m².

### Example 4

Absorbent forming the absorptive core of a sanitary aid is, before applying into the absorptive core, homogenized with a complex of polyvinylpyrolidone (PVP)-hydrogen peroxide encapsulated by an encapsulation agent magnesium stearate. The ratios for encapsulation are 2:1 (encapsulation agent:reactive form of oxygen), the encapsulation was carried out by a melting granulation in the ratio of 40 parts by weight of absorbent and 1 weight part of PPV-hydrogen peroxide. Such prepared mixture is used for the production of an absorbent core of a menstrual pad.

### Example 5

Absorbent forming the absorptive core of a sanitary aid is, before applying into the absorptive core, homogenized with a complex of (PVP)-hydrogen peroxide encapsulated by bee wax (ratios for encapsulation 2:1—encapsulation agent:reactive form of oxygen) in the ratio of 100 parts by weight of absorbent and 1 weight part of the complex of PVP-hydrogen peroxide. Such prepared mixture was enriched by 1 weight part of sodium perborate and is used for the production of an absorbent core of a menstrual pad.

### Example 6

Absorbent forming the absorptive core of a sanitary aid is, before applying into the absorptive core, homogenized with a complex of polyvinylpyrolidone (PVP)-hydrogen peroxide encapsulated by magnesium stearate (ratios for encapsulation 2:1—encapsulation agent: reactive form of oxygen) in the ratio of 40 parts by weight of absorbent and 1 part by weight of the encapsulated complex of PVP-hydrogen peroxide and 0.1 part by weight of non-encapsulated, that is non-encapsulated polyvinylpyrolidon (PVP)-hydrogen peroxide. Such prepared mixture is used for the production of an absorbent core of a menstrual pad in the amount of 1 g per pad.

### Example 7

Superabsorbent forming the absorptive core of a sanitary aid is, before applying into the absorptive core, homogenized with a complex of polyvinylpyrolidon (PVP)-hydrogen peroxide encapsulated by a combination of glyceryl monostearate and gylceryl monolaurate (ratios for encapsulation 1:1:1—encapsulation agent I:encapsulation agent II:reactive form of oxygen) in the ratio of 40 parts by weight of superabsorbent and 1 part by weight of encapsulated complex of PVP-hydrogen peroxide. Such prepared mixture is used for the production of an absorbent core of a menstrual pad.

### Example 8

Absorbent forming the absorptive core of a sanitary aid is, before applying into the absorptive core, homogenized with a complex PVP-hydrogen peroxide in the ratio of 40 parts by weight of absorbent and 3 parts by weight of the complex of PVP-hydrogen peroxide and 0.2 parts by weight of encapsulated sodium perborate. Sodium perborate is encapsulated by carnauba wax in the weight ratio of 25% sodium perborate and 75% carnauba wax. The encapsulation takes place by intensive mixing of the wax warmed at 70° C. with sodium perborate. Such prepared mixture is used for the production of an absorbent core of a menstrual pad. The amount applied in the pad core is 2 g of the final mixture.

### Example 9

Absorbent forming the absorptive core of a sanitary aid is, before applying into the absorptive core, homogenized with a complex of PVP-hydrogen peroxide in the ratio of 40 parts by weight of absorbent and 3 parts by weight of the complex of PVP-hydrogen peroxide encapsulated by monoglyceride (encapsulation by melt granulation of the mixture of 70% by weight of monoglyceride and 30% by weight of PVP-hydrogen peroxide) and 1 part by weight of the sodium hydrogencarbonate. Such prepared mixture is used for the production of an absorbent core of a menstrual pad.

### Example 10

Absorbent forming the absorptive core of a sanitary aid is, before applying into the absorptive core, homogenized with a complex of sodium peroxide in the ratio of 40 parts by weight of absorbent and 3 parts by weight of sodium peroxide encapsulated by microcrystalline cellulose in the form of dry granulation of a mixture of 10% by weight of sodium peroxide and 90% of microcrystalline cellulose. Such prepared mixture is used for the production of an absorbent core of an incontinence napkin. The amount applied into the absorbent core of the napkin is 1 to 10 g of such prepared superabsorbent mixture.

### Example 11

Absorbent forming the absorptive core of a sanitary aid is, before applying into the absorptive core, homogenized with a complex of PVP-hydrogen peroxide in the ratio 40 parts by weight of absorbent and 3 parts by weight of the complex of magnesium peroxide encapsulated by shellac and 0.1 parts by weight of sodium hydrogencarbon for the adjustment of pH. Such prepared mixture is used for the production of an absorbent core of a sanitary incontinence napkin in the amount of 0.1 to 15 g or for applying on the inner side of the topsheet in the concentration of 0.5 per 1 m².

### Example 12

Absorbent forming the absorptive core of a sanitary aid is, before applying into the absorptive core, homogenized with a complex of PVP-hydrogen peroxide in the ratio 80 parts by weight of the absorbent and 3 parts by weight of the complex of PVP-hydrogen peroxide encapsulated by melt granulation by monoglyceride and subsequently with the added 1 part by weight of monolaurin and 1 part by weight of crystalline lactic acid. Such prepared mixture is used for the production of an absorbent core of a baby diaper.

### Example 13

At the topsheet layer of a feminine menstrual pad the amount of 0.1 g of powder is applied by a roller with a complex of PVP-hydrogen peroxide encapsulated by monoglyceride (encapsulation on fragmentation grinder 66.6% by weight of glyceryl monostearate and 33.4% by weight of PVP-hydrogen peroxide) in the mixture with mannitol in the ratio 1:9. The mixture is prepared in the ratio 10 parts by weight of mannitol and 1 part by weight of encapsulated complex of PVP-hydrogen peroxide. The absorbent core of a pad is enriched by aloe vera extract.

### Example 14

At the topsheet layer of a feminine menstrual pad it is applied by a roller 0.1 g of powder with a complex of calcium peroxide encapsulated by monolaurin (encapsulation by melt granulation of the mixture in parts by weight of calcium peroxide:monolaurin in the ratio 1:10) in the mixture with mannitol in the ratio of 1 part by weight of reactive form of oxygen: 9 parts by weight of mannitol. The absorbent core of a pad is enriched by a probiotic element, i.e. inulin from chicory in the amount of 0.1 g per pad.

### Example 15

At the topsheet layer of a feminine menstrual pad it is applied by a roller 0.1 g of powder with a complex of PVP-hydrogen peroxide encapsulated by stearane in the mixture with isomalt in the ratio 1:9. The mixture is prepared in the ratio of 10 parts by weight of isomalt and 1 part by weight of encapsulated complex of PVP-hydrogen peroxide. The absorbent core of a pad is enriched by 100 mg of cranberry extract.

### Example 16

At the topsheet layer of a feminine menstrual pad it is applied by a roller 0.1 g of powder with a complex of PVP-hydrogen peroxide encapsulated by monoglyceride and monolaurin and stearane in the weight ratio 1:2:1 in the mixture with microcrystalline cellulose in the ratio 1:9. The mixture is prepared in the ratio of 10 parts by weight of microcrystalline cellulose a 1 part by weight of encapsulated complex of PVP-hydrogen peroxide. The absorbent core of a pad is enriched by 50 mg of tea tree extract.

### Example 17

At the topsheet layer of a feminine menstrual pad it is applied by a roller 0.1 g of powder with a complex of sodium perborate encapsulated by a combination of monoglyceride, monolaurin and stearane in the mixture with mannitol in the ratio 1:9. The mixture is prepared in the ratio of 10 parts by weight of mannitol and 1 part by weight of complex of sodium perborate.

### Example 18

At the topsheet layer of a sanitary incontinency pad or napkin it is applied by a roller 0.1 g of powder with a complex of sodium perborate encapsulated by monoglyceride in the mixture with sorbitol. The mixture is prepared in the ratio of 8 parts by weight of sorbitol and 1 part by weight of sodium perborate.

### Example 19

At the topsheet layer of a feminine menstrual pad it is applied by a roller 0.1 g of powder with a complex of PVP-hydrogen peroxide encapsulated by shellac (encapsulation by PVP-hydrogen peroxide 1 part by weight:shellac 2 parts by weight, encapsulation by grinding at a fragmentation grinder) and a mixture of probiotic lyophilized culture Lactobacillus acidophilus or Bacillus coagulans in the amount of 1 billion CFU per pad.

### Example 20

At the topsheet layer of a feminine menstrual pad it is applied by a roller 0.1 g of powder with a complex of PVP-hydrogen peroxide encapsulated by monoglyceride and monolaurin with an addition of probiotic lyophilized culture Lactobacillus acidophilus and a prebiotic element in the form of a plant soluble fiber-13 inulin or corn soluble fiber in the amount of 0.12 g per pad.

### Example 21

At the topsheet layer of a feminine menstrual pad it is applied by a roller 0.12 g of powder with a complex of PVP-hydrogen peroxide encapsulated by a mixture of microcrystalline cellulose or MCC with sunflower oil (MCC 94% by weight:sunflower oil 6% by weight)—encapsulation by dry granulation 1 part of PVP-hydrogen peroxide and 4 parts of a mixture of MCC with sunflower oil with an addition of extract from chamomile in the amount of 0.05 g per pad.

### Example 22

At the topsheet layer of a feminine menstrual pad it is applied by a roller or a nozzle 0.05 g of powder with a complex of PVP-hydrogen peroxide encapsulated by monoglyceride in the mixture with non-encapsulated sodium perborate-calcium peroxide.

### Example 23

At the inner layer of topsheet of an incontinence napkin it is applied by a roller or a nozzle 0.1 g of powder with the complex of PVP-hydrogen peroxide encapsulated in an extruder by monoglyceride and monolaurin (weight ratio 1:2:1) in the mixture with non-encapsulated PVP-hydrogen peroxide.

Example 24

At the topsheet layer of the feminine menstrual pad it is applied by a roller 0.08 g of powder with a complex of PVP-hydrogen peroxide encapsulated by monoglyceride in the mixture of monoglyceride encapsulated by sodium perborate.

### Example 25

Absorbent forming the absorptive core of a baby diaper is, before applying into the napkin core, homogenized with a complex of PVP-hydrogen peroxide encapsulated by dry granulation by monoglyceride and monolaurin and stearane in the weight ratio 1:1:1:1. Such prepared mixture is used for the production of an absorbent core of a baby diaper.

### Example 26

Absorbent forming the absorptive core of a sanitary incontinence aid is, before applying into the absorptive core, homogenized with a complex of PVP-hydrogen peroxide and sodium perboritane (1:1) encapsulated by monostearate 1:3. Such prepared mixture is used for the production of an absorbent core in a mixture with superabsorbent (5% mixture), whereas it is possible to add 1% by weight of sodium perboritane monohydrate, which may be micronized before application.

### Example 27

Absorbent forming the absorptive core of a sanitary incontinence aid is, before applying into the absorptive core, homogenized with a complex of PVP-hydrogen peroxide encapsulated by stearane and carboxymethylcelullose in the ratio 15:1:3. Such prepared mixture may be further combined with sodium perborate.

### Example 28

In the cellulose absorbent forming the absorptive core of a sanitary incontinency aid in a form of panty liner is apply a homogenate of the complex of PVP-hydrogen peroxide encapsulated by monoglyceride amonolaurin and hydroxymethylpropylcellulose in the ratio 3:8 is applied. Such prepared mixture is used for the production of the absorbent core of a panty liner.

### Example 29

Absorbent forming the absorptive core of a sanitary aid is, before applying into the absorptive core, homogenized with a complex of sodium peroxide encapsulated by a melt granulation by glyceryl monostearate in the ratio of 30 parts by weight of absorbent and 1 part by weight of the complex of PVP-hydrogen peroxide and 1 part by weight of microcrystalline cellulose and 0.00001 parts by weight silver. Such prepared mixture is used for the production of the absorbent core of a menstrual pad, in the optimum amount of 1 to 3 g per pad.

### Example 30

At the surface layer of toilet paper it is applied by a roller 0.1 g power with the complex of PVP-hydrogen peroxide encapsulated by monoglyceride and monolaurin in the mixture with marigold extract.

### Example 31

At the surface layer of toilet paper it is applied by a roller 0.02 g power with the complex of PVP-hydrogen peroxide encapsulated by monoglyceride in the mixture with sorbitol. The mixture is prepared in the ratio of 6 parts by weight of sorbitol and 1 part by weight of the encapsulated complex of PVP-hydrogen peroxide.

### Example 32

Between layers of a paper tissue it is applied by a roller 0.01 g of powder with a complex of percarbonate encapsulated by melt granulation by monoglyceride and monolaurin in the ratio 1:3:2 in the mixture with tea tree and eucalypt extract. The mixture is prepared in the ratio of 2 parts by weight of encapsulated complex and 1 part by weight of plant extract and 2 parts by weight of microcrystalline cellulose. The mixture can be added also to unprocessed cellulose in the first step of the tissue production.

### Example 33

The absorbent forming the absorptive core of a sanitary aid is, before applying into the absorptive core, homogenized with the complex of sodium perborate encapsulated by magnesium stearane (the weight ratios for encapsulation 3:1—encapsulation agent:reactive form of oxygen) in the ratio of 60 parts by weight of absorbent and 1 part by weight of encapsulated sodium perborate. To such prepared mixture it may be added 0.1 parts by weight of crystalline lactic acid. The mixture is used for the production of the absorbent core of a menstrual pad. The amount of 1.2 g of the mixture is applied in the absorptive core.

### INDUSTRIAL APPLICABILITY

The sanitary aid with antibacterial effect for stabilization of physiological balance in vaginal environment and/or external genitalia based on this invention can be used particularly in feminine hygiene in the form of menstrual pads, towels or napkins, daily and overnight pads, liners and tampons usable both at the time of menstruation as well as in the time between menstruations to ensure a quality feminine intimacy hygiene with the objective to stabilize physiological balance in vaginal environment and/or external genitalia. Furthermore, the sanitary aid can be used in routine baby hygiene in case of baby diapers as well as in incontinence aids, or toilet paper and paper tissues.

1. A sanitary aid with antibacterial effect for stabilization of physiological balance in vaginal environment and/or external genitalia made by a backsheet impermeable layer, absorbent core formed by a mixture of absorbent and/or superabsorbent and a topsheet layer from woven or non-woven textile or from cellulose, containing an effective component on the basis of at least one reactive form of oxygen, which is arranged on the topsheet layer of a sanitary aid designated for the contact with body and/or in the absorbent core, characterized in that the reactive forms of oxygen are contained in the effective substance in the form encapsulated by an encapsulation agent, ensuring gradual release of reactive forms of oxygen after contact with liquid from the sanitary aid.

2. The sanitary aid according to claim 1, characterized in that the reactive form of oxygen is in the form of complex of organic peroxide, particularly polyvinylpyrolidon-hydrogen peroxide or in the form of inorganic compounds of hydrogen peroxide, particularly magnesium peroxide, calcium peroxide, sodium percarbonate and sodium perborate or their mutual combinations.

3. The sanitary aid according to claim 1, characterized in that the reactive form of oxygen is encapsulated by an encapsulation agent from the group: monolaurin, monoglycerides, stearanes, glyceryles, stearates, sterols, laurels, laureates and other derivatives of fatty acids, microcrystalline cellulose, hydroxypropylmethyl cellulose, omega-3 fatty acids, mannitol, sorbitol, xylitol, isomalt, humin compounds, sunflower oil, rapeseed oil, olive oil, carnauba wax, bee wax, phospholipids, lactic and whey proteins, casein, gelatin, gluten, paraffin, shellac or their mutual combinations.

4. The sanitary aid according to claim 1, characterized in that the reactive form of oxygen is encapsulated in the ration 0.01 to 99 parts by weight of the reactive form of oxygen and 0.1 to 99.9 parts of the encapsulation agent, with advantage 1 up to 5 parts by weight of the reactive form of oxygen and 2 to 20 parts by weight of the encapsulation agent.

5. The sanitary aid according to claim 1, characterized in that the effective substance is in the form of micronized particles with the size from 1 to 999 μm, with an advantage from 1 to 100 μm.

6. The sanitary aid according to claim 1, characterized in that the topsheet layer contains a mixture formed by 0.01 to 99.99 parts by weight, with an advantage 50 to 90 parts by weight of the absorbent or superabsorbent and 0.01 to 50 parts by weight, with an advantage 5 to 20 parts by weight of an effective substance.

7. The sanitary aid according to claim 1, characterized in that the absorbent core contains a mixture formed by 1 to 99.999 parts by weight, with an advantage 50 to 99.9 parts by weight of the absorbent or superabsorbent and 0.001 to 50 parts by weight, with an advantage 0.1 to 3 parts by weight of an effective substance.

8. The sanitary aid according to claim 1, characterized in that it further contains freely arranged reactive forms of oxygen at the topsheet layer of the sanitary aid designated for contact with body and/or in the internal structure of the sanitary aid.

9. The sanitary aid according to claim 1, characterized in that it further contains an auxiliary substance at the topsheet layer of the sanitary aid designated for contact with body and/or in the internal structure of the sanitary aid selected from the group: lactic acid, its pharmaceutically acceptable salt, lyophilized probiotic culture, prebiotic element, plant extracts, with an advantage herb extracts, titanium dioxide, antimicrobial metals, with an advantage silver, or their mutual mixtures.

10. The sanitary aid based on claim 1, characterized in that it is made in the form of menstrual pads, towels or napkins, or daytime or overnight pads, or panty liners, or baby diapers, or incontinence pads or napkins, or tampons, or toilet paper, or paper tissues.
